(19) United States
(12) Patent Application Publication
(10) Pub. No.: US 2009/0118265 A1
(43) Pub. Date: May 7, 2009

(54) NOVEL 1,4-DIAZA-BICYCLO[3.2.2]NONANE DERIVATIVES AND THEIR MEDICAL USE

(76) Inventors: Dan Peters, Malmo (SE); Gunnar M. Olsen, Smorum (DK); Elsebet Ostergaard Nielsen, Kobenhavn (DK); Daniel B. Timmermann, Herlev (DK); Steven Charles Loechel, Frederiksberg (DK); Jeppe Kejser Christensen, Kobenhavn (DK); Tino Dyhring, Solrod (DK)

Correspondence Address: BIRCH STEWART KOLASCH & BIRCH, PO BOX 747, FALLS CHURCH, VA, 22040-0747, US

(21) Appl. No.: 12/300,010

(22) PCT Filed: May 21, 2007
(86) PCT No.: PCT/EP07/54869

§ 371 Date: Nov. 7, 2008

Related U.S. Application Data
(60) Provisional application No. 60/802,528, filed on May 23, 2006.

(30) Foreign Application Priority Data
May 23, 2006 (DK) PA 2006 00704

(51) Int. Cl. A61K 31/5513; C07D 471/08; A61P 17/00; A61P 17/10; A61P 15/12; A61P 15/10; A61P 15/06; A61P 11/06; A61P 1/06; A61P 1/00; A61P 25/14; A61P 25/24; A61P 25/18; A61P 25/22; A61P 25/06; A61P 25/28; A61P 25/00; A61P 25/36; A61P 25/34; A61P 25/32; A61P 9/12; A61P 17/06; A61P 25/16; A61P 3/04

(52) U.S. Cl. 514/221; 540/556

(57) ABSTRACT

This invention relates to novel 1,4-diaza-bicyclo[3.2.2]nonane derivatives, which are found to be cholinergic ligands at the nicotinic acetylcholine receptors and modulators of the monoamine receptors and transporters. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel 1,4-diaza-bicyclo[3.2.2]nonane derivatives, which are found to be cholinergic ligands at the nicotinic acetylcholine receptors and modulators of the monoamine receptors and transporters. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism. Indeed several CNS disorders can be attributed to a cholinergic deficiency, a dopaminergic deficiency, an adrenergic deficiency or a serotonergic deficiency.

### SUMMARY OF THE INVENTION

The present invention is devoted to the provision novel modulators of the nicotinic and/or of the monoamine receptors, which modulators are useful for the treatment of diseases or disorders related to the cholinergic receptors, and in particular the nicotinic acetylcholine receptor (nAChR), the serotonin receptor (5-HTR), the dopamine receptor (DAR) and the norepinephrine receptor (NER), and of the biogenic amine transporters for serotonin (5-HT), dopamine (DA) and norepinephrine (NE).

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides novel 1,4-diaza-bicyclo[3.2.2]-nonane derivatives represented by Formula I

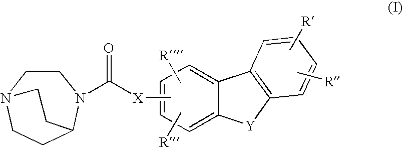

or a pharmaceutically acceptable salt thereof, wherein

X represents O or NH;

Y represents O, S, CO, SO, SO₂, CH₂, CHOH or C═N—OZ, wherein Z represents hydrogen or alkyl; and

R′, R″, R′″ and R″″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

In its second aspect the invention provides pharmaceutical compositions comprising a therapeutically effective amount of the 1,4-diaza-bicyclo[3.2.2]nonane derivatives of the invention, or a pharmaceutically-acceptable addition salt thereof, or a prodrug thereof, together with at least one pharmaceutically-acceptable carrier or diluent.

In a further aspect the invention relates to the use of the 1,4-diaza-bicyclo[3.2.2]nonane derivatives of the invention, or a pharmaceutically-acceptable addition salt thereof, for the manufacture of a pharmaceutical composition/-medicament for the treatment, prevention or alleviation of a disease or a disorder or a condition of a mammal, including a human, which disease, disorder or condition is responsive to modulation of cholinergic receptors and/or monoamine receptors.

In a final aspect the invention provides methods of treatment, prevention or alleviation of diseases, disorders or conditions of a living animal body, including a human, which disorder, disease or condition is responsive to modulation of cholinergic receptors and/or monoamine receptors, which method comprises the step of administering to such a living animal body in need thereof a therapeutically effective amount of the 1,4-diaza-bicyclo[3.2.2]nonane derivatives of the invention.

Other objects of the invention will be apparent to the person skilled in the art from the following detailed description and examples.

### DETAILED DISCLOSURE OF THE INVENTION

### 1,4-Diaza-Bicyclo[3.2.2]Nonane Derivatives

In a first aspect novel 1,4-diaza-bicyclo[3.2.2]nonane derivatives are provided. The 1,4-diaza-bicyclo[3.2.2]nonane derivatives of the invention may be represented by Formula I

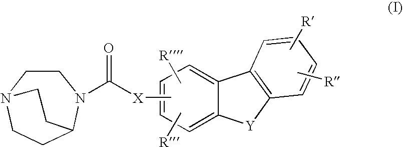

or a pharmaceutically acceptable salt thereof, wherein

X represents O or NH;

Y represents O, S, CO, SO, SO₂, CH₂ CHOH or C═N—OZ, wherein Z represents hydrogen or alkyl; and

R′, R″, R′″ and R″″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

In a first more preferred embodiment the 1,4-diaza-bicyclo[3.2.2]nonane derivative of the invention is a compound of Formula Ia

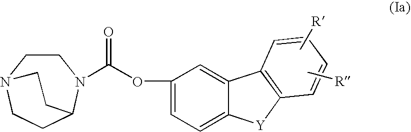

or a pharmaceutically acceptable salt thereof, wherein

X represents O or NH;

Y represents O, S, CO, SO, SO₂, CH₂, CHOH or C═N—OZ, wherein Z represents hydrogen or alkyl; and

R′ and R″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

In a second more preferred embodiment the 1,4-diaza-bicyclo[3.2.2]nonane derivative of the invention is a compound of Formula Ib

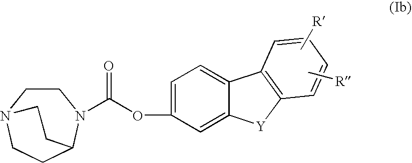

or a pharmaceutically acceptable salt thereof, wherein

X represents O or NH;

Y represents O, S, CO, SO, SO₂, CH₂, CHOH or C═N—OZ, wherein Z represents hydrogen or alkyl; and

R′ and R″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

In a third more preferred embodiment the 1,4-diaza-bicyclo[3.2.2]nonane derivative of the invention is a compound of Formula I, Ia or Ib, wherein X represents O or NH.

In a more preferred embodiment X represents O.

In another more preferred embodiment X represents NH.

In a fourth more preferred embodiment the 1,4-diaza-bicyclo[3.2.2]nonane derivative of the invention is a compound of Formula I, Ia or Ib, wherein Y represents O, S, CO, SO, SO₂, CH₂, CHOH or C═N—OZ, wherein Z represents hydrogen or alkyl.

In an even more preferred embodiment Y represents O, S, CO, SO₂, CH₂ or CHOH.

In a still more preferred embodiment Y represents O, CO, CH₂ or CHOH.

In a further more preferred embodiment Y represents O.

In a still further more preferred embodiment Y represents S.

In a still further more preferred embodiment Y represents CO.

In a still further more preferred embodiment Y represents SO₂.

In a still further more preferred embodiment Y represents CH₂.

In a still further more preferred embodiment Y represents CHOH.

In a fifth more preferred embodiment the 1,4-diaza-bicyclo[3.2.2]nonane derivative of the invention is a compound of Formula I, Ia or Ib, wherein

R′, R″, R′″ and R″″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

In an even more preferred embodiment R′, R″, R′″ and R″″, independently of each other, represent hydrogen, alkyl, cycloalkyl, hydroxy, alkoxy, halo or trifluoromethyl.

In a still more preferred embodiment R′ and R″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl; and R′″ and R″″ both represent hydrogen.

In a yet more preferred embodiment R′ and R″, independently of each other, represent hydrogen, alkyl, cycloalkyl, hydroxy, alkoxy, halo or trifluoromethyl; and R′″ and R″″ both represent hydrogen.

In a further more preferred embodiment R′ represents hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl; and R″, R′″ and R″″ all represent hydrogen.

In a still further more preferred embodiment R′ represents hydrogen, alkyl, cycloalkyl, hydroxy, alkoxy, halo or trifluoromethyl; and R′, R′″ and R″″ all represent hydrogen.

In a still further more preferred embodiment R′, R″, R′″ and R″″ all represent hydrogen.

In a most preferred embodiment the 1,4-diaza-bicyclo[3.2.2]nonane derivative of the invention is

1,4-Diaza-bicyclo[3.2.2]nonane-4-carboxylic acid 9-oxo-9H-fluoren-2-yl ester;

1,4-Diaza-bicyclo[3.2.2]nonane-4-carboxylic acid 9H-fluoren-2-yl ester;

1,4-Diaza-bicyclo[3.2.2]nonane-4-carboxylic acid dibenzofuran-3-yl ester;

1,4-Diaza-bicyclo[3.2.2]nonane-4-carboxylic acid (9H-fluoren-2-yl)-amide;

1,4-Diaza-bicyclo[3.2.2]nonane-4-carboxylic acid 9-hydroxy-9H-fluoren-2-yl ester;

1,4-Diaza-bicyclo[3.2.2]nonane-4-carboxylic acid dibenzothiophen-4-yl ester;

1,4-Diaza-bicyclo[3.2.2]nonane-4-carboxylic acid 5,5-dioxo-dibenzothiophen-4-yl ester; or

1,4-Diaza-bicyclo[3.2.2]nonane-4-carboxylic acid dibenzofuran-4-yl ester; or a pharmaceutically acceptable salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention a cycloalkyl-alkyl group designates a cycloalkyl group as defined above, which cycloalkyl group is substituted on an alkyl group as also defined above. Examples of preferred cycloalkyl-alkyl groups of the invention include cyclopropylmethyl and cyclopropylethyl.

In the context of this invention an alkoxy group designates an “alkyl-O—” group, wherein alkyl is defined as a univalent saturated, straight or branched hydrocarbon chain, preferably containing of from one seven carbon atoms (C₁₋₇-alkoxy). Examples of preferred alkoxy groups of the invention include methoxy and ethoxy.

In the context of this invention an alkyl-carbonyl group designates an “alkyl-CO— group”, wherein alkyl is as defined above. Examples of preferred alkyl-carbonyl groups of the invention include acetyl and propionyl.

In the context of this invention an alkoxy-carbonyl group designates an “alkyl-O—CO—” group, wherein alkyl is as defined above. Examples of preferred alkoxy-carbonyl groups of the invention include the methyl-, ethyl- and propyl-ester group.

In the context of this invention an alkyl-carbonyl-oxy group designates an “alkyl-CO—O—” group, wherein alkyl is as defined above. Examples of preferred alkyl-carbonyl-oxy groups of the invention include acetoxy and propionyloxy.

In the context of this invention halo represents fluoro, chloro, bromo or iodo. Thus a trihalomethyl group represents e.g. a trifluoromethyl group, a trichloromethyl group, and similar trihalo-substituted methyl groups.

In the context of this invention a haloalkyl group designates an alkyl group as defined herein, which alkyl group is substituted one or more times with halo. Preferred haloalkyl groups of the invention include trihalomethyl, preferably trifluoromethyl.

In the context of this invention a haloalkoxy group designates an alkoxy group as defined herein, which alkoxy group is substituted one or more times with halo. Preferred haloalkoxy groups of the invention include trihalomethoxy, preferably trifluoromethoxy.

### Pharmaceutically Acceptable Salts

The 1,4-diaza-bicyclo[3.2.2]nonane derivative of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a chemical compound of the invention include alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radio-nuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), and combinations thereof.

### Methods of Producing 1,4-Diaza-Bicyclo[3.2.2]Nonane Derivatives

The 1,4-diaza-bicyclo[3.2.2]nonane derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel ligands and modulators of the nicotinic receptors, which ligands and modulators are useful for the treatment of diseases or disorders related to the cholinergic receptors, and in particular the nicotinic acetylcholine receptor (nAChR). Preferred compounds of the invention show a pronounced nicotinic acetylcholine α7 receptor subtype selectivity.

Due to their pharmacological profile the compounds of the invention may be useful for the treatment of diseases or conditions as diverse as CNS related diseases, PNS related diseases, diseases related to smooth muscle contraction, endocrine disorders, diseases related to neuro-degeneration, diseases related to inflammation, pain, and withdrawal symptoms caused by the termination of abuse of chemical substances.

In a preferred embodiment the compounds of the present invention may be useful for the treatment, prevention or alleviation of a cognitive disorder, learning deficit, memory deficits and dysfunction, Down's syndrome, Alzheimer's disease, attention deficit, attention deficit hyperactivity disorder (ADHD), Tourette's syndrome, psychosis, depression, Bipolar Disorder, mania, manic depression, schizophrenia, cognitive or attention deficits related to schizophrenia, obsessive compulsive disorders (OCD), panic disorders, eating disorders such as anorexia nervosa, bulimia and obesity, narcolepsy, nociception, AIDS-dementia, senile dementia, autism, Parkinson's disease, Huntington's disease, Amyotrophic Lateral Sclerosis, anxiety, non-OCD anxiety disorders, convulsive disorders, epilepsy, neurodegenerative disorders, transient anoxia, induced neuro-degeneration, neuropathy, diabetic neuropathy, periferic dyslexia, tardive dyskinesia, hyperkinesia, mild pain, moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury, bulimia, post-traumatic syndrome, social phobia, sleeping disorders, pseudodementia, Ganser's syndrome, pre-menstrual syndrome, late luteal phase syndrome, fibromyalgia, chronic fatigue syndrome, mutism, trichotillomania, jet-lag, arrhythmias, smooth muscle contractions, angina pectoris, premature labour, diarrhoea, asthma, tardive dyskinesia, hyperkinesia, premature ejaculation, erectile difficulty, hypertension, inflammatory disorders, inflammatory skin disorders, acne, rosacea, Chron's disease, inflammatory bowel disease, ulcerative colitis, diarrhoea, or withdrawal symptoms caused by termination of use of addictive substances, including nicotine containing products such as tobacco, opioids such as heroin, cocaine and morphine, benzodiazepines and benzodiazepine-like drugs, and alcohol.

In a more preferred embodiment the compounds of the invention may be useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In an even more preferred embodiment the compounds of the invention may be useful for the treatment, prevention or alleviation of diseases, disorders or conditions associated with smooth muscle contractions, convulsive disorders, angina pectoris, premature labour, convulsions, diarrhoea, asthma, epilepsy, tardive dyskinesia, hyperkinesia, premature ejaculation, or erectile difficulty.

In a still more preferred embodiment the compounds of the invention may be useful for the treatment, prevention or alleviation of a neurodegenerative disorder, transient anoxia, or induced neuro-degeneration.

In a yet more preferred embodiment the compounds of the invention may be useful for the treatment, prevention or alleviation of an inflammatory disorder, inflammatory skin disorder, acne, rosacea, Chron's disease, inflammatory bowel disease, ulcerative colitis, or diarrhoea.

In a further preferred embodiment the compounds of the invention may be useful for the treatment, prevention or alleviation of diabetic neuropathy, schizophrenia, cognitive or attentional deficits related to schizophrenia, or depression.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines, benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

In another aspect, the compounds of the invention are used as diagnostic agents, e.g. for the identification and localisation of nicotinic receptors in various tissues.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i e. of from below 1 to about 100 μM.

### Pharmaceutical Compositions

In another aspect the invention provides novel pharmaceutical compositions comprising a therapeutically effective amount of the 1,4-diaza-bicyclo[3.2.2]nonane derivatives, derivative of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the 1,4-diaza-bicyclo[3.2.2]nonane derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The 1,4-diaza-bicyclo[3.2.2]nonane derivatives of the present invention are valuable nicotinic and monoamine receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors and/or monoamine receptors, and which method comprises administering to such a living animal body, including a human, in need thereof an effective amount of a 1,4-diaza-bicyclo[3.2.2]nonane derivative of the invention.

In a preferred embodiment, the disease, disorder or condition relates to the central nervous system.

The preferred medical indications contemplated according to the invention are those stated above.

It is at present contemplated that suitable dosage ranges are within 0.1 to 1000 milligrams daily, preferably 10 to 500 milligrams daily, and more preferred of from 30 to 100 milligrams daily, dependent as usual upon the exact mode of administration, form in which administered, the indication toward which the administration is directed, the subject involved, the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Reparatory Example

All reactions involving air sensitive reagents or intermediates were performed under nitrogen and in anhydrous solvents. Magnesium sulfate was used as drying agent in the workup-procedures and solvents were evaporated under reduced pressure.

1,4-Diazabicyclo[3.2.2]Nonane (Intermediate Compound)

The title compound was prepared according to J. Med. Chem. 1993 36 2311-2320 (and according to a slightly modified method below).

1,4-Diazabicyclo[3.2.2]Nonane (Intermediate Compound)

To the solution of 1,4-diazabicyclo[3.2.2]nonan-3-one (15.8 g; 113 mmol) in absolute dioxane (130 ml) LiAIH₄ (4.9 g; 130 mmol) was added under argon. The mixture was refluxed for 6 h and then allowed to reach room temperature. To the reaction mixture water (5 ml in 10 ml of dioxane) was added by drops, the mixture was stirred for 0.5 hour and then filtered off via glass filter. The solvent was evaporated and the residue was distilled using Kugelrohr apparatus at 90° C. (0.1 mbar) to yield 1,4-diazabicyclo[3.2.2]nonane (11.1 g, 78%) as colourless hygroscopic material.

1,4-Diazabicyclo[3.2.2]Nonan-3-One (Intermediate Compound)

To the solution of 3-quinuclidinone hydrochloride (45 g; 278 mmol) in 90 ml of water hydroxylamine hydrochloride (21 g; 302 mmol) and sodium acetate (CH₃COOHx3H₂O; 83 g; 610 mmol) were added, the mixture was stirred at 70° C. for 1 hour and then cooled to 0° C. The separated crystalline material was filtered off (without washing) and dried in vacuo to yield 40.0 g of oxime.

The 3-quinuclidinone oxime (40.0 g) was added during 2 hours by small portions to preheated to 120° C. polyphosphoric acid (190 g). The temperature of the solution during the reaction was kept at 130° C. After addition of all oxime the solution was stirred for 20 minutes at the same temperature, then transferred to an enamelled vessel and allowed to reach room temperature. The acidic mixture was neutralized by a solution of potassium carbonate (500 g in 300 ml of water), transferred into 2000 ml flask, diluted with 300 ml of water and extracted with chloroform (3 ×600 ml). The combined organic extracts were dried with sodium sulphate, the solvent evaporated and the solid residue dried up in vacuo to yield 30.0 g (77%) of the mixture of lactams.

Crystallization of the obtained mixture from 1,4-dioxane (220 ml) gave 15.8 g (40.5%) of 1,4-diazabicyclo[3.2.2]nonan-3-one as colourless large crystals with mp. 211-212° C.

The filtrate was evaporated and the residue was chromatographed on a silica gel (Merck, 9385, 230-400 mesh) column with acetone as eluent. The solvent was evaporated and the residue recrystallized from ethyl etanoate to yield 1,3-diazabicyclo[3.2.2]nonan-4-one (10.2 g; 26%) as colourless fine crystals with mp. 125-126° C.

### Method A

### 1,4-Diaza-Bicyclo[3.2.2]Nonane-4-Carboxylic Acid 9-Oxo-9H-Fluoren-2-yl Ester Fumaric Acid Salt (Compound A1)

Phosgen, 20% in toluene (2.87 g, 29.0 mmol) was solved in anhydrous dichloromethane (15 ml) at 0° C. A mixture of 2-hydroxy-fluoren-9-one (1.14 g, 5.81 mmol) and pyridine (0.61 g, 7.73 mmol), solved in dichloromethane (25 ml) was added to the mixture at 0° C. The mixture was stirred for 30 minutes at 0° C. and was then allowed to reach room-temperature and was stirred over-night. The reaction-mixture was evaporated and was co-evaporated with toluene (25 ml). The crude intermediate, 1,4-diazabicyclo[3.2.2]nonane (0.73 g 5.81 mmol) and 1,2-dimethoxyethane (40 ml) was stirred at room-temperature for 6 days. Aqueous sodium hydroxide (50 ml, 1 M) was added followed by extraction with chloroform (3×30 ml). Chromatography on silica gel with chloroform, 10% methanol and 1% aqueous ammonia as solvent gave the title compound as an oil. The corresponding salt was obtained by addition of a diethyl ether and methanol mixture (9:1) saturated with fumaric acid. Yield 1.40 g, 0.51%. LC-ESI-HRMS of [M+H]+ shows 349.1562 Da. Calc. 349.155218 Da, dev. 2.8 ppm.

### 1,4-Diaza-Bbicyclo[3.2.21]Nonane-4-Carboxylic Acid 9H-Fluoren-2-yl Ester Fumaric Acid Salt (Compound A2)

Was prepared from 9H-fluoren-3-ol according to Method A. LC-ESI-HRMS of [M+H]+ shows 335.1768 Da. Calc. 335.175953 Da, dev. 2.5 ppm.

### 1,4-Diaza-Bicyclo[3.2.2]Nonane-4-Carboxylic Acid Dibenzofuran-3-yl Ester Fumaric Acid Salt (Compound A3)

Was prepared from dibenzofuran-3-ol according to Method A. LC-ESI-HRMS of [M+H]+ shows 337.1549 Da. Calc. 337.155218 Da, dev. −0.9 ppm.

### 1,4-Diaza-Bicyclo[3.2.2]Nonane-4-Carboxylic Acid Dibenzothiophen-4-yl Ester Fumaric Acid Salt (Compound A4)

Was prepared from dibenzothiophen-4-ol according to Method A. LC-ESI-HRMS of [M+H]+ shows 353,1329 Da. Calc. 353,132374 Da, dev. 1,5 ppm.

### 1,4-Diaza-Bicyclo[3.2.2]Nonane-4-Carboxylic Acid 5,5-Dioxo-Dibenzothiophen-4-yl Ester Fumaric Acid Salt (Compound A5)

Was prepared from 5,5-dioxo-dibenzothiophen-4-ol according to Method A. LC-ESI-HRMS of [M+H]+ shows 385,1221 Da. Calc. 385,122204 Da, dev. −0,3 ppm.

### 1,4-Diaza-Bicyclo[3.2.2]Nonane-4-Carboxylic Acid Dibenzofuran-4-yl Ester Fumaric Acid Salt (Compound A6)

Was prepared from dibenzofuran-4-ol according to Method A. LC-ESI-HRMS of [M+H]+ shows 337.1556 Da. Calc. 337.155218 Da, dev. 1.1 ppm.

### Method B

1,4-Diaza-Bicyclo[3.2.2]Nonane-4-Carboxylic Acid (9H-Fluoren-2-yl)-Amide Fumaric Acid Salt (Compound B1)

A mixture of 1,4-diazabicyclo[3.2.2]nonane (2.0 g, 15.8 mmol) and methanol (40 ml) was stirred at −50° C. 9-H-fluoren-2-yl-isocyanate (4.3 g, 20.6 mmol) was added at −50° C. The reaction-mixture was allowed to stir at room-temperature for 3 days. Aqueous sodium hydroxide (50 ml, 1 M) was added followed by extraction with chloroform (3×50 ml). Chromatography on silica gel with chloroform, 10% methanol and 1% aqueous ammonia as solvent gave the title compound as an oil. The corresponding salt was obtained by addition of a diethyl ether and methanol mixture (9:1) saturated with fumaric acid. Yield 2.04 g, 28%. LC-ESI-HRMS of [M+H]+ shows 334.1919 Da. Calc. 334.191937 Da, dev. −0.1 ppm

### Method C

### 1.4-Diaza-Bicyclo[3.2.21]Nonane-4-Carboxylic Acid 9-Hydroxy-9H-Fluoren-2-yl Ester Free Base (Compound C1)

A mixture of 1,4-diaza-bicyclo[3.2.2]nonane-4-carboxylic acid 9-oxo-9H-fluoren-2-yl ester (0.53 g, 1.51 mmol), sodium borohydride (85 mg, 2.26 mmol) and methanol (20 ml) was allowed to stir for 1 hour at room temperature. The mixture was evaporated. Aqueous sodium hydroxide (20 ml, 1 M) was added followed by extraction by chloroform (3×20 ml). Chromatography on silica gel with dichloromethane, 10% methanol and 1% aqueous ammonia as solvent gave the title compound as a solid. Yield 0.53 g (100%). LC-ESI-HRMS of [M+H]+ shows 334.1919 Da. Calc. 334.191937 Da, dev. −0.1 ppm.

### Method D

### Dibenzothiophen-4-ol (Intermediate Compound)

A mixture of 4-dibenzothiopheneboronic acid (2.5 g, 11.0 mmol), hydrogen peroxide (2.5 ml, 35%) and ethanol (30 ml, 96%) was stirred at reflux for 2 h. The ethanol was evaporated. Water (50 ml) was added and the mixture was extracted with ethylacetate (2×50 ml). Chromatography on silica gel with petroleum and ethyl acetate as solvent gave the title compound. Yield 1.1 g (50%).

### Dibenzofuran-4-ol (Intermediate Compound)

Was prepared according to Method D from 4-dibenzofuranboronic acid.

### Method E

### 5,5-Dioxo-dibenzothiophen-4-ol (Intermediate Compound)

A mixture of 4-dibenzothiopheneboronic acid (2.6 g, 11.4 mmol), mCPBA (11.2 g, 45.6 mmol), ethanol (10 ml) and chloroform (25 ml) was stirred at 20° C. for 15 h. Water (50 ml) was added and the mixture was extracted with ethylacetate (2×50 ml). Chromatography on silica gel with dichloromethane, 10% methanol and 1% aqueous ammonia as solvent gave the title compound as a solid. Yield 1.66 g (63%).

1-10. (canceled)

11. A 1,4-diaza-bicyclo[3.2.2]nonane derivative represented by Formula I 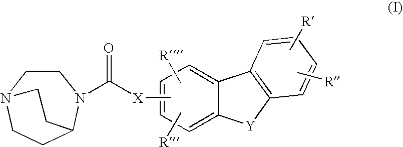
or a pharmaceutically acceptable salt thereof, wherein
X represents O or NH;
Y represents O, S, CO, SO, SO₂, CH₂, CHOH or C═N—OZ, wherein Z represents hydrogen or alkyl; and
R′, R″, R′″ and R″″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

12. The 1,4-diaza-bicyclo[3.2.2]nonane derivative of claim 11, or a pharmaceutically acceptable salt thereof, wherein X represents O or NH.

13. The 1,4-diaza-bicyclo[3.2.2]nonane derivative of claim 11, or a pharmaceutically acceptable salt thereof, wherein Y represents O, S, CO, SO, SO₂, CH₂, CHOH or C═N—OZ, wherein Z represents hydrogen or alkyl.

14. The 1,4-diaza-bicyclo[3.2.2]nonane derivative of claim 11, or a pharmaceutically acceptable salt thereof, wherein R′, R″, R′″ and R″″, independently of each other, represent hydrogen, alkyl, cycloalkyl, cycloalkyl-alkyl, hydroxy, alkoxy, halo, haloalkyl, haloalkoxy, cyano, nitro, amino, oxo, carboxy, alkyl-carbonyl, alkoxy-carbonyl, alkyl-carbonyl-oxy, carbamoyl, amido, sulfamoyl, phenyl or benzyl.

15. The 1,4-diaza-bicyclo[3.2.2]nonane derivative of claim 14, or a pharmaceutically acceptable salt thereof wherein R′, R″, R′″ and R″″ all represent hydrogen.

16. The 1,4-diaza-bicyclo[3.2.2]nonane derivative of claim 11, which is
1,4-Diaza-bicyclo[3.2.2]nonane-4-carboxylic acid 9-oxo-9H-fluoren-2-yl ester;
1,4-Diaza-bicyclo[3.2.2] nonane-4-carboxylic acid 9H-fluoren-2-yl ester;
1,4-Diaza-bicyclo[3.2.2]nonane-4-carboxylic acid dibenzofuran-3-yl ester;
1,4-Diaza-bicyclo[3.2.2]nonane-4-carboxylic acid (9H-fluoren-2-yl)-amide;
1,4-Diaza-bicyclo[3.2.2]nonane-4-carboxylic acid 9-hydroxy-9H-fluoren-2-yl ester;
or a pharmaceutically acceptable salt thereof.

17. A pharmaceutical composition comprising a therapeutically effective amount of the 1,4-diaza-bicyclo[3.2.2]nonane derivative of claim 11, or a pharmaceutically-acceptable salt thereof, together with at least one pharmaceutically-acceptable carrier or diluent.

18. A method of treatment, prevention or alleviation of a disease or a disorder or a condition of a living animal body, including a human, which disorder, disease or condition is responsive to modulation of cholinergic receptors and/or monoamine receptors, which method comprises the step of administering to such a living animal body in need thereof a therapeutically effective amount of the 1,4-diaza-bicyclo[3.2.2]nonane derivative of claim 11 or a pharmaceutically acceptable salt thereof.

19. The method according to claim 18, wherein the disease, disorder or condition is a cognitive disorder, learning deficit, memory deficits and dysfunction, Down's syndrome, Alzheimer's disease, attention deficit, attention deficit hyperactivity disorder (ADHD), Tourette's syndrome, psychosis, depression, Bipolar Disorder, mania, manic depression, schizophrenia, cognitive or attention deficits related to schizophrenia, obsessive compulsive disorders (OCD), panic disorders, eating disorders such as anorexia nervosa, bulimia and obesity, narcolepsy, nociception, AIDS-dementia, senile dementia, autism, Parkinson's disease, Huntington's disease, Amyotrophic Lateral Sclerosis, anxiety, non-OCD anxiety disorders, convulsive disorders, epilepsy, neurodegenerative disorders, transient anoxia, induced neuro-degeneration, neuropathy, diabetic neuropathy, periferic dyslexia, tardive dyskinesia, hyperkinesia, mild pain, moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury, bulimia, post-traumatic syndrome, social phobia, sleeping disorders, pseudodementia, Ganser's syndrome, pre-menstrual syndrome, late luteal phase syndrome, fibromyalgia, chronic fatigue syndrome, mutism, trichotillomania, jet-lag, arrhythmias, smooth muscle contractions, angina pectoris, premature labour, diarrhoea, asthma, tardive dyskinesia, hyperkinesia, premature ejaculation, erectile difficulty, hypertension, inflammatory disorders, inflammatory skin disorders, acne, rosacea, Chron's disease, inflammatory bowel disease, ulcerative colitis, diarrhoea, or withdrawal symptoms caused by termination of use of addictive substances, including nicotine containing products such as tobacco, opioids such as heroin, cocaine and morphine, benzodiazepines and benzodiazepine-like drugs, and alcohol.
